(19) United States
(12) Patent Application Publication
(10) Pub. No.: US 2019/0008819 A1
(43) Pub. Date: Jan. 10, 2019

(54) Methods and Compositions Using Cetyl Myristoleate (CMO) In Medical Treatments

(71) Applicant: Bruce H. Levin, Oceanside, NY (US)

(72) Inventor: Bruce H. Levin, Oceanside, NY (US)

(21) Appl. No.: 16/028,371

(22) Filed: Jul. 5, 2018

Related U.S. Application Data
(60) Provisional application No. 62/529,721, filed on Jul. 7, 2017.

(51) Int. Cl. A61K 31/231; A61P 25/02; A61P 25/06; A61K 9/70

(52) U.S. Cl. CPC A61K 31/231; A61P 25/02; A61K 45/06; A61K 9/7038; A61K 2300/00; A61P 25/06

(57) ABSTRACT

A therapeutic method of alleviating the symptoms of failed back surgery syndrome, post laminectomy syndrome, post-surgery syndrome, sympathetic dystrophies, macular degenerations, complex regional pain syndrome, reflex sympathetic dystrophy and neuropathies by administering to the afflicted subject a therapeutically effective amount of cetyl myristoleate alone, or in combination with selective effective amount of aspirin, gabapentin, pregabalin, duloxetine, selective serotonin reuptake inhibitors (SSRIs), tricyclic antidepressants, neuroceuticals, Neurontin, Cymbalta, Lyrica, muscle relaxers, anti-depressants, nortriptyline, local anesthetics, and steroids.

### RELATED APPLICATIONS

This application claims priority to U.S. Provisional Patent Application Ser. No. 62/529,721, filed Jul. 7, 2017, titled the same and incorporated herein as if set out in full.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### FIELD OF THE INVENTION

The present invention relates to the therapeutic method useful in treating and alleviating symptoms or pathologies of failed back surgery syndrome, post laminectomy syndrome, post-surgery syndromes, sympathetic dystrophies, macular degenerations, complex regional pain syndrome, reflex sympathetic dystrophy and neuropathies, small fiber neuropathies, metabolic neuropathies, diabetic or endocrine neuropathies, post-therapeutic neuropathy, peripheral neuropathies, cranial neuropathies, radiculopathies, migraine headache, cluster headache, chronic headache, daily headache or tension headache, facial pain, or TMD.

### BACKGROUND OF THE INVENTION

Cetyl myristoleate (CMO) is the common name for cis-9-Cetyl myristoleate. CMO is a fatty acid ester that are naturally present in mice that are immune to arthritis, even when researchers tried to induce arthritis in the mice. CMO has been associated with blocking inflammation, lubricating joints and muscles, softening tissues, and increasing flexibility. It has been used to treat arthritis and joint pain.

Arthritis is a disease of inflammation in the joints causing joint pain and stiffness. Because of CMO's property to decrease inflammation and prevent arthritis, CMO has been used to treat arthritis and joint pain. However, the exact mechanism of how CMO actions is unclear, and CMO has not be widely used to treat many diseases.

### SUMMARY OF THE INVENTION

The present invention describes a formula comprising a proper dosage of cetyl myristoleate for treating diseases and aliments such as failed back surgery syndrome, post laminectomy syndrome, post-surgery syndromes, sympathetic dystrophies, macular degenerations, complex regional pain syndrome, reflex sympathetic dystrophy and neuropathies, small fiber neuropathies, metabolic neuropathies, diabetic or endocrine neuropathies, post-therapeutic neuropathy, peripheral neuropathies, cranial neuropathies, radiculopathies, migraine headache, cluster headache, chronic headache, daily headache or tension headache, facial pain, and TMD.

Furthermore, the formula may be used in combination with one or more of the following: aspirin, gabapentin, pregabalin, duloxetine, selective serotonin reuptake inhibitors (SSRIs), tricyclic antidepressants, neuroceuticals, Neurontin, Cymbalta, Lyrica, muscle relaxants, anti-depressants, nortriptyline, CGRP agents, agonists or antagonists, local anesthetics, and steroids.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

The invention disclosed herein relates to the use of cetyl myristoleate (CMO) and CMO compounds in combination with other compounds useful for treating disease and other ailments. Cetyl myristoleate compounds have herein been found to be particularly useful when used in combination with components of accepted therapies. When used in this manner, cetyl myristoleate compounds can provide significant symptomatic relief.

In one embodiment, the present invention provides a formula comprising administering 0.1 milligrams to 10 grams of cetyl myristoleate once to four times per day for treating diseases and aliments such as failed back surgery syndrome, post laminectomy syndrome, post-surgery syndrome, sympathetic dystrophies, macular degenerations, complex regional pain syndrome, reflex sympathetic dystrophy and neuropathies.

For the above embodiment, the CMO may be used in combination with one or more of the following: aspirin, gabapentin, pregabalin, duloxetine, selective serotonin reuptake inhibitors (SSRIs), tricyclic antidepressants, neuroceuticals, Neurontin, Cymbalta, Lyrica, muscle relaxers, anti-depressants, nortriptyline, local anesthetics, and steroids.

In another embodiment, the present invention provides a formula comprising administering 0.1 milligrams to 10 grams of cetyl myristoleate once to four times per day for decreasing the destructiveness of failed back surgery syndrome, post laminectomy syndrome, post-surgery syndrome, sympathetic dystrophies, macular degenerations, complex regional pain syndrome, reflex sympathetic dystrophy and neuropathies.

For the above embodiment, the CMO may be used in combination with one or more of the following: aspirin, gabapentin, pregabalin, duloxetine, selective serotonin reuptake inhibitors (SSRIs), tricyclic antidepressants, neuroceuticals, Neurontin, Cymbalta, Lyrica, muscle relaxers, anti-depressants, nortriptyline, local anesthetics, and steroids.

In another embodiment, the present invention provides a formula comprising administering 0.1 milligrams to 10 grams of cetyl myristoleate once to four times per day for decreasing the progression of failed back surgery syndrome, post laminectomy syndrome, post-surgery syndromes, sympathetic dystrophies, macular degenerations, complex regional pain syndrome, reflex sympathetic dystrophy and neuropathies.

For the above embodiment, the CMO may be used in combination with one or more of the following: aspirin, gabapentin, pregabalin, duloxetine, selective serotonin reuptake inhibitors (SSRIs), tricyclic antidepressants, neuroceuticals, Neurontin, Cymbalta, Lyrica, muscle relaxers, anti-depressants, nortriptyline, CGRP agents, agonists or antagonists, local anesthetics, and steroids.

For the above embodiment, the CMO may be used in combination with one or more of the following: Anti Nerve Growth Factor agents, including but not limited to monoclonal antibodies such as Tanezumab, and/or Anti Calcitonin Gene Related peptide monoclonal antibodies can be directly injected or infused into or around spinal structures including discs, ligaments, tendons, facet joints, muscles, nerve roots, neuroforamen, epidurally or intrathecally, or into or around sympathetic nervous system plexuses or nerve structures, peripheral nerves, or directly into joints or intranasally, to decrease symptoms or pathologies of the referenced conditions, alone or in combination with CMO and/or the classes of agents herein noted. In addition, combining CMO or chondroitin Sulfate to AntiNGF agents will decrease joint degeneration in the setting of arthritis. Other agents include ketamine, NMDA antagonist, i.e. Dextromethorphan.

In another embodiment, an effective amount of cetyl myristoleate is administered between 12 grams to 18 grams over a period of a month to treat failed back surgery syndrome, post laminectomy syndrome, post-surgery syndrome, sympathetic dystrophies, macular degenerations, complex regional pain syndrome, reflex sympathetic dystrophy and neuropathies.

In another embodiment, an effective amount of cetyl myristoleate is administered between 0.1 milligrams to 10 grams for once to four times per day to treat failed back surgery syndrome, post laminectomy syndrome, post-surgery syndrome, sympathetic dystrophies, macular degenerations, complex regional pain syndrome, reflex sympathetic dystrophy and neuropathies.

In another embodiment, the invention provides administering cetyl myristoleate through a transdermal delivery device comprising a backing layer and a matrix layer underlying the backing layer. The matrix layer of the transdermal delivery device comprises a mixture of cetyl myristoleate and a pressure sensitive adhesive.

In another embodiment, the transdermal delivery device wherein the device is worn for between 5 and 10 days.

In another embodiment, the invention provides the transdermal delivery device wherein the cetyl myristoleate is delivered between 0.01 mg/kg/day and 10 mg/kg/day.

In another embodiment, the transdermal delivery device wherein the matrix layer further comprises one or more of the components selected from the group consisting of glucosamine sulfate, chondroitin sulfate, sea cucumber extract, hydrolyzed shark cartilage, collagen II, and methylsulfonylmethane.

In another embodiment, the invention provides a method of treating a disease associated with the inflammation of tissues.

In an embodiment, the transdermal delivery device comprising cetyl myristoleate is affixed to the skin of a mammal such as an animal or human.

Alternatively, in another embodiment, Dimethyl sulfoxide (DMSO) or similar epithelial transport compound is applied to aid delivery transdermal.

In another embodiment, the invention provides an oral medicament comprising cetyl myristoleate and an enteric coating. The enteric coating is resistant to dissolution in the stomach but predisposed to dissolution in the intestine so as to prevent release of the cetyl myristoleate until the medicament is in the intestine.

In another embodiment, the invention provides for an oral medicament comprising cetyl myristoleate and an enteric coating. The enteric coating is resistant to dissolution in an environment having a pH less than 5.5.

In a further embodiment, the invention provides for an oral medicament comprising cetyl myristoleate and an enteric coating. The oral medicament comprises between 0.1 gram and 1 gram of cetyl myristoleate. Alternatively, a suppository comprising cetyl myristoleate may be administered to a human or animal to treat hemorrhoid discomfort, perianal irritation, rectal discomfort, or any other disorder mentioned in this application.

### Example 1

A middle age male with complex regional pain syndrome had post-surgery pain. It was so severe that the male was to have foot amputation for the pain. A dosage of 0.1 milligrams to 10 grams of cetyl myristoleate once to four times per day was administered to the male. Shortly, the male described the pain decreased by 20-30 percent. The patient has not undergone amputation.

### Example 2

A middle age male was taking a brief CMO regime for knee pain. When administering 0.1 milligrams to 10 grams of cetyl myristoleate once to four times per day with appropriate dosage gabapentin, the knee pain decreased dramatically.

### Example 3

A middle age male was taking a brief CMO regime for shoulder pain. When administering 0.1 milligrams to 10 grams of cetyl myristoleate once to four times per day with an effective dosage gabapentin, the shoulder pain decreased dramatically.

### Example 4

A middle age female experienced cervical post-surgery syndrome. After administering 0.1 milligrams to 10 grams of cetyl myristoleate once to four times per day, the post-laminectomy syndrome (PLS) was improved significantly.

It will be apparent that topical preparations may further comprise substances which enhance absorption. Such absorption enhancers are well known to those of ordinary skill in the art. Examples include but are not limited to dimethyl sulfoxide (DMSO), fatty acids, micelles, and microsome and liposome preparations.

Compositions can be administered by a variety method which are well known in the art. Routes of administration include, but are not limited to oral, topical, sublingual, rectal, intranasal, intraocular, intravenous, intramuscular, transdermal, and by inhalation.

However, for delivery to specific sites of inflammation, other means can be used for administering the composition such as, for example, by intraarticular, periarticular or intraosseous injection. Delivery methods can employ microsomes or liposomes. Where desirable, active components can be formulated into timed-release products.

There is no requirement that active components used in treatment be administered by the same route or at the same time. For example, in one embodiment, a tetracycline compound is administered orally according to a first schedule and CMO compound is administered orally according to a second schedule.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A method for reducing a pain producing condition in a mammal comprising administering 0.1 milligrams to 10 grams of cetyl myristoleate once to four times per day.

2. The method of claim 1 wherein said condition is failed back surgery syndrome, post laminectomy syndrome, post-surgery syndromes, sympathetic dystrophies, macular degenerations, complex regional pain syndrome, reflex sympathetic dystrophy and neuropathies, small fiber neuropathies, metabolic neuropathies, diabetic or endocrine neuropathies, post-therapeutic neuropathy, peripheral neuropathies, cranial neuropathies, radiculopathies, migraine headache, cluster headache, chronic headache, daily headache or tension headache, facial pain, or TMD.

3. The method of claim 1 wherein administering 0.1 milligrams to 10 grams of cetyl myristoleate once to four times per day is used in combination with one or more of the following: aspirin, gabapentin, pregabalin, duloxetine, selective serotonin reuptake inhibitors (SSRIs), tricyclic antidepressants, nutraceuticals, Neurontin, Cymbalta, Lyrica, muscle relaxants, anti-depressants, CGRP agents, agonists or antagonists, nortriptyline, local anesthetics, and steroids.

4. A method for reducing a pain producing condition in a mammal comprising administering 0.1 milligrams to 10 grams of cetylated fatty acid once to four times per day.

5. The method of claim 4 wherein said condition is failed back surgery syndrome, post laminectomy syndrome, post-surgery syndromes, sympathetic dystrophies, macular degenerations, complex regional pain syndrome, reflex sympathetic dystrophy and neuropathies, small fiber neuropathies, metabolic neuropathies, diabetic or endocrine neuropathies, post-therapeutic neuropathy, peripheral neuropathies, cranial neuropathies, radiculopathies, migraine headache, cluster headache, chronic headache, daily headache or tension headache, facial pain, or TMD.

6. The method of claim 4 wherein administering 0.1 milligrams to 10 grams of cetyl myristoleate once to four times per day is used in combination with one or more of the following: aspirin, gabapentin, pregabalin, duloxetine, selective serotonin reuptake inhibitors (SSRIs), tricyclic antidepressants, nutraceuticals, Neurontin, Cymbalta, Lyrica, muscle relaxants, anti-depressants, CGRP agents, agonists or antagonists, nortriptyline, local anesthetics, and steroids.

7. A method of reduce knee pain or shoulder pain of degenerative, post traumatic, overuse or other etiologies or of any arthropathy or tendinopathy, comprising administering to a patient in need thereof a compound cetyl myristoleate, or a pharmaceutically acceptable cetylated fatty acid thereof, and gabapentin, or other neuropathic agents or pharmaceuticals thereof in amounts that in combination are effective.

8. The method of claim 7 wherein said condition is failed back surgery syndrome, post laminectomy syndrome, post-surgery syndromes, sympathetic dystrophies, macular degenerations, complex regional pain syndrome, reflex sympathetic dystrophy and neuropathies, small fiber neuropathies, metabolic neuropathies, diabetic or endocrine neuropathies, post-therapeutic neuropathy, peripheral neuropathies, cranial neuropathies, radiculopathies, migraine headache, cluster headache, chronic headache, daily headache or tension headache, facial pain, or TMD.

9. The method of claim 7 wherein administering 0.1 milligrams to 10 grams of cetyl myristoleate once to four times per day is used in combination with one or more of the following: aspirin, gabapentin, pregabalin, duloxetine, selective serotonin reuptake inhibitors (SSRIs), tricyclic antidepressants, nutraceuticals, Neurontin, Cymbalta, Lyrica, muscle relaxants, anti-depressants, CGRP agents, agonists or antagonists, nortriptyline, local anesthetics, and steroids.

10. The method of claim 1 wherein the cetyl myristoleate is delivered through a transdermal device.

11. The method of claim 4 wherein the cetyl myristoleate is delivered through a transdermal device.

12. The method of claim 7 wherein the cetyl myristoleate is delivered through a transdermal device.

13. The method of claim 10 wherein said transdermal device further comprises
a. a backing layer, and
b. a matrix layer underlying the backing layer wherein the matrix layer of the transdermal delivery device further comprises a mixture of cetyl myristoleate and a pressure sensitive adhesive.

14. The matrix layer of claim 13 further comprises one or more of the components selected from the group consisting of glucosamine sulfate, chondroitin sulfate, sea cucumber extract, hydrolyzed shark cartilage, collagen II, and methylsulfonylmethane.

15. The method of claim 14 wherein said transdermal device is worn for between 5 to 10 days.

16. The method of claim 14 wherein between 0.01 mg/kg/day and 10 mg/kg/day of cetyl myristoleate is delivered.

17. The method of claim 11 wherein said transdermal device further comprises
a. a backing layer, and
b. a matrix layer underlying the backing layer wherein the matrix layer of the transdermal delivery device further comprises a mixture of cetyl myristoleate and a pressure sensitive adhesive.

18. The method of claim 17 wherein said transdermal device is worn for between 5 to 10 days.

19. The method of claim 17 wherein between 0.01 mg/kg/day and 10 mg/kg/day of cetyl myristoleate is delivered.

20. The method of claim 12 wherein said transdermal device further comprises
a. a backing layer, and
b. a matrix layer underlying the backing layer wherein the matrix layer of the transdermal delivery device further comprises a mixture of cetyl myristoleate and a pressure sensitive adhesive.

21. The method of claim 12 wherein said transdermal device is worn for between 5 to 10 days.

22. The method of claim 12 wherein between 0.01 mg/kg/day and 10 mg/kg/day of cetyl myristoleate is delivered.
